(19) United States
(12) Patent Application Publication
(10) Pub. No.: US 2026/0157633 A1
(43) Pub. Date: Jun. 11, 2026

(54) WEARABLE DEVICE, SYSTEM, AND METHOD FOR MONITORING SOFT-TISSUE YOUNG'S MODULUS

(71) Applicants: Suzhou Institute for Advanced Research, University of Science and Technology of China, Suzhou (CN); University of Science and Technology of China, Hefei (CN)

(72) Inventors: Tingrui PAN, Suzhou (CN); Huizhi WANG, Suzhou (CN); Yu CHANG, Suzhou (CN); Zhenning WANG, Suzhou (CN)

(73) Assignees: Suzhou Institute for Advanced Research, University of Science and Technology of China, Suzhou (CN); University of Science and Technology of China, Hefei (CN)

(21) Appl. No.: 19/299,377

(22) Filed: Aug. 14, 2025

(30) Foreign Application Priority Data
Dec. 11, 2024 (CN) 202411814270.6

(51) Int. Cl. A61B 5/00; A61B 90/00

(52) U.S. Cl. CPC A61B 5/0053; A61B 5/6831; A61B 5/7271; A61B 90/06; A61B 2090/064; A61B 2562/0247; A61B 2562/046; A61B 2562/227

(57) ABSTRACT

A wearable monitoring device for soft-tissue Young's modulus includes a pressure application mechanism, a contact radius sensor, a contact force sensor, and a wearable mechanism. The pressure application mechanism includes an indenter with a hemispherical contact surface. The contact radius sensor includes a flexible pressure sensing array disposed on the contact surface and configured to detect a pressure distribution along a height direction of the indenter. The contact radius sensor is configured to output a first electrical signal in response to a contact pressure when the contact surface contacts a measured soft tissue. The contact force sensor is disposed on the pressure application mechanism and configured to output a second electrical signal in response to the contact pressure provided by the pressure application mechanism when the contact surface contacts the measured soft tissue. The monitoring device can achieve accurate, continuous, and dynamic monitoring of the Young's modulus of soft tissues.

### CROSS REFERENCE TO THE RELATED APPLICATIONS

This application is based upon and claims priority to Chinese Patent Application No. 202411814270.6, filed on Dec. 11, 2024, the entire contents of which are incorporated herein by reference.

### TECHNICAL FIELD

The present disclosure belongs to the technical field of biomedical engineering, and in particular relates to a wearable device, system, and method for monitoring soft-tissue Young's modulus.

### BACKGROUND

Palpation is a typical traditional method for detecting soft tissue hardness. For example, clinicians subjectively assess changes in tissue hardness by pressing on the lesion tissue with their fingers to determine disease progression. However, palpation is highly subjective, lacks quantitative metrics, and cannot achieve continuous and dynamic monitoring. Some traditional mechanical hardness detection systems, such as universal tensile testing machines, calculate the Young's modulus of the measured object by recording a variation curve of the deformation of the measured object versus the applied force. However, these systems rely on large hardware equipment, require the measured object to have standard shape and size, and cannot achieve dynamic monitoring of human soft tissue modulus. Ultrasound elastography detects the modulus by analyzing the deformation of the measured object when pressed or wave propagation speed within the measured object. Such techniques can utilize handheld or wearable devices to support dynamic detection. However, limited by complex factors like tissue heterogeneity and thickness, along with high operator dependence, they generally only provide relative modulus reference values and face significant challenges in providing accurate absolute values. Indentation methods based on contact mechanics theories like the Hertzian model can calculate the Young's modulus of the measured object by detecting the pressure and the indentation depth/contact radius generated when a hemispherical probe is pressed into the soft measured object. This calculation process does not require the measured object to have standard shape and size, enabling rapid development and widespread application of related technologies like atomic force microscopes (AFMs) and nanoindenters. However, since indentation measurement requires precise force and displacement control and feedback systems, it is generally confined to large hardware facilities and cannot achieve dynamic and continuous monitoring of human soft tissues.

Chinese Patent Application CN115500811A proposes a tactile sensor based on dual closed-loop control and a control method thereof. The tactile sensor includes a dual closed-loop control system, a sensor measurement system, and a tactile sensor system model. The dual closed-loop control system includes a sensor probe, an amplitude-stabilizing control system, and a frequency tracking system. The sensor measurement system includes a pressing device and a resonance frequency acquisition system. The tactile sensor system model is derived based on the control model of the tactile sensor and the equivalent acoustic impedance when the sensor probe contacts the target tissue, and is configured to calculate the elasticity of the target tissue by tracking the frequency shift of a self-excited oscillation circuit. This system can improve measurement accuracy, reduce the system resonance frequency, and avoid invasive damage to the target tissue. However, it requires precise control of contact force magnitude and stable contact conditions during use, as well as coordinated operation of the dual closed-loop control system, resulting in complex structure, and does not support continuous monitoring.

Chinese Patent Application CN116685839A proposes a modulus sensor, including a base, a sensor, an indenter, and a locking device. The sensor is fixedly connected to the base. The indenter is slidably connected to the base to move axially relative to the base in response to a first contact between the indenter and a material surface, thereby providing a thrust to the sensor in the axial direction. The locking device is configured to lock the indenter in a releasable locked state in response to a second contact between the base and the material surface, where the indenter in the locked state is prevented from moving axially relative to the base. When a thrust is applied continuously, the indenter is locked by the locking device at a certain position. At this point, the sensor measures the thrust and the displacement of the indenter. Then, based on a Hertzian model, the Young's modulus is calculated. However, this device requires the construction of a locking mechanism, resulting in a complex structure, and cannot achieve continuous and dynamic monitoring.

In summary, existing technologies for monitoring the Young's modulus of soft tissues still face significant challenges in achieving continuous, dynamic, and accurate monitoring.

### SUMMARY

In view of this, the present disclosure provides a wearable device, system, and method for monitoring soft-tissue Young's modulus. The present disclosure can achieve accurate, continuous, and dynamic monitoring of the Young's modulus of soft tissues based on a Hertzian contact model, utilizing flexible tactile sensing technology integrating a dual-modal (contact force and contact radius) detection solution.

A first aspect of the present disclosure provides a wearable device for monitoring soft-tissue Young's modulus, including a pressure application mechanism, a contact radius sensor, a contact force sensor, and a wearable mechanism, where the pressure application mechanism includes an indenter, and the indenter is provided with a hemispherical contact surface; the wearable mechanism is configured to carry the pressure application mechanism and to bring the contact surface into contact with a measured soft tissue when the wearable mechanism is worn; the contact radius sensor includes a flexible pressure sensing array disposed on the contact surface and configured to detect a pressure distribution along a height direction of the indenter; the contact radius sensor is configured to output a first electrical signal in response to a contact pressure when the contact surface contacts the measured soft tissue; and the contact force sensor is disposed on the pressure application mechanism and configured to output a second electrical signal in response to the contact pressure provided by the pressure application mechanism when the contact surface contacts the measured soft tissue.

A second aspect of the present disclosure provides a wearable system for monitoring soft-tissue Young's modulus, including a host computer, and the wearable device for monitoring soft-tissue Young's modulus according to the first aspect of the present disclosure, and

alternatively, including the host computer, a data acquisition device, and the wearable device for monitoring soft-tissue Young's modulus according to the first aspect of the present disclosure, where the data acquisition device is communicatively connected to the host computer; and the contact radius sensor and the contact force sensor are electrically connected to the data acquisition device via wires, respectively; and

the host computer includes a calculation module, a storage module, and a communication module; the calculation module is configured to call a program pre-stored in the storage module and execute a following instruction: acquiring the first electrical signal and the second electrical signal received by the communication module; calculating a contact radius between the contact surface and the measured soft tissue based on the first signal; converting the second electrical signal into a contact force between the contact surface and the measured soft tissue; and calculating an equivalent Young's modulus value of the measured soft tissue based on a Hertzian equation, the contact radius and the contact force.

A third aspect of the present disclosure provides a method for wearably monitoring soft-tissue Young's modulus, including:
acquiring a first electrical signal output by a flexible pressure sensing array in a contact radius sensor in response to a contact pressure when a contact surface of the pressure application mechanism contacts a measured soft tissue, where the contact surface is hemispherical; and the flexible pressure sensing array is disposed on the contact surface and configured to detect a pressure distribution along a height direction of an indenter;
calculating a contact radius between the contact surface and the measured soft tissue based on the first electrical signal;
acquiring a second electrical signal output by a contact force sensor in response to the contact pressure provided by the pressure application mechanism when the contact surface of the pressure application mechanism contacts the measured soft tissue, where the contact force sensor is disposed on the pressure application mechanism;
converting the second electrical signal into a contact force between the contact surface and the measured soft tissue; and
calculating an equivalent Young's modulus value of the measured soft tissue based on a Hertzian equation, the contact force and the contact radius.

The present disclosure has the following beneficial effects:
(1) The present disclosure adopts a wearable manner. The present disclosure achieves simultaneous and continuous detection of the contact force and contact radius between the indenter and the measured soft tissue through the contact force sensor and the contact radius sensor. The contact force sensor is provided on the pressure application mechanism. The contact radius sensor is attached to the indenter contact surface of the pressure application mechanism and includes the flexible pressure sensing array. Moreover, this wearable manner no longer requires the measured object to be stationary, thereby achieving wearable continuous and dynamic monitoring of the soft-tissue Young's modulus.
(2) The present disclosure achieves highly accurate detection of the contact radius through the flexible pressure array sensor featuring a high spatial resolution and a low detection limit. In particular, the present disclosure uses the contact radius sensor based on an iontronic principle to more readily achieve low-threshold response and high-spatial-resolution arrangement of sensing units in the sensing array, thereby enabling accurate monitoring of the soft-tissue Young's modulus.
(3) The present disclosure provides multiple design solutions for the contact radius sensor, which can be selected based on actual needs.
(4) The present disclosure can assist in quantitatively evaluating disease progression, monitoring soft tissue functions during daily activities, optimizing athletic performance, avoiding athletic injuries, and providing quantitative evidence for rehabilitation training.

### BRIEF DESCRIPTION OF THE DRAWINGS

FIG. 1 is a stereoscopic view of a wearable device for monitoring soft-tissue Young's modulus;

FIG. 2 is a bottom view of the wearable device for monitoring soft-tissue Young's modulus;

FIG. 3 is a front view of the wearable device for monitoring soft-tissue Young's modulus;

FIG. 4 is an A-A sectional view of the wearable device for monitoring soft-tissue Young's modulus;

FIG. 5 is a B-B sectional view of the wearable device for monitoring soft-tissue Young's modulus;

FIG. 6 is a C-C sectional view of the wearable device for monitoring soft-tissue Young's modulus;

FIG. 7 is a D-D sectional view of the wearable device for monitoring soft-tissue Young's modulus;

FIG. 8 is an internal structural schematic diagram (within a housing) of the wearable device for monitoring soft-tissue Young's modulus;

FIG. 9 is a schematic diagram of a flat pattern of a contact radius sensor;

FIGS. 10A-10D are other structural schematic diagrams of the contact radius sensor, respectively;

FIG. 11 is a schematic diagram of a system working principle;

FIG. 12 is a schematic diagram of a contact radius calculation principle;

FIG. 13 is a schematic diagram of a measurement state; and

FIGS. 14A-14C are schematic diagrams respectively showing temporal variations in contact force, contact radius, and Young's modulus of the tissue at the gastrocnemius muscle site during stair ascent.

Reference Numerals: 1. indenter; 101. contact surface; 102. top surface; 2. contact radius sensor; 201. sensing unit; 3. contact force sensor; 4. housing; 401. base; 402. top cover; 403. screw hole; 404. wire outlet; 405 (405a, 405b, 405c). strap groove; 5. extension column; 6. flange; 7. wire; and 8. strap.

### DETAILED DESCRIPTION OF THE EMBODIMENTS

The technical solutions of the present disclosure will be clearly and completely described below in conjunction with specific embodiments and drawings of the present disclosure. Obviously, the described embodiments are only a part, rather than all of the embodiments of the present disclosure. All other embodiments obtained by those skilled in the art based on the embodiments of the present disclosure without creative efforts should fall within the protection scope of the present disclosure.

In the description of the present disclosure, orientation or position relationships indicated by terms such as “upper”, “lower”, “front”, “rear”, “inside”, and “outside” are based on the drawings. These terms are merely intended to facilitate description of the present disclosure and simplify the description, rather than to indicate or imply that the mentioned device or element must have a specific orientation and be constructed and operated in a specific orientation. Therefore, these terms should not be construed as a limitation to the present disclosure. Those of ordinary skill in the art may understand specific meanings of the foregoing terms in the present disclosure based on a specific situation. In addition, the terms “including” and “having” and any variations thereof are intended to cover non-exclusive inclusions that may include other units not clearly listed or inherent to these products or devices.

In addition, in the drawings of the embodiments provided in the present disclosure, the connection relationship between modules or components represents a communicative connection between the modules or components, which may be specifically implemented by one or more communication buses or signal lines. Those of ordinary skill in the art can understand and implement the embodiments without creative effort.

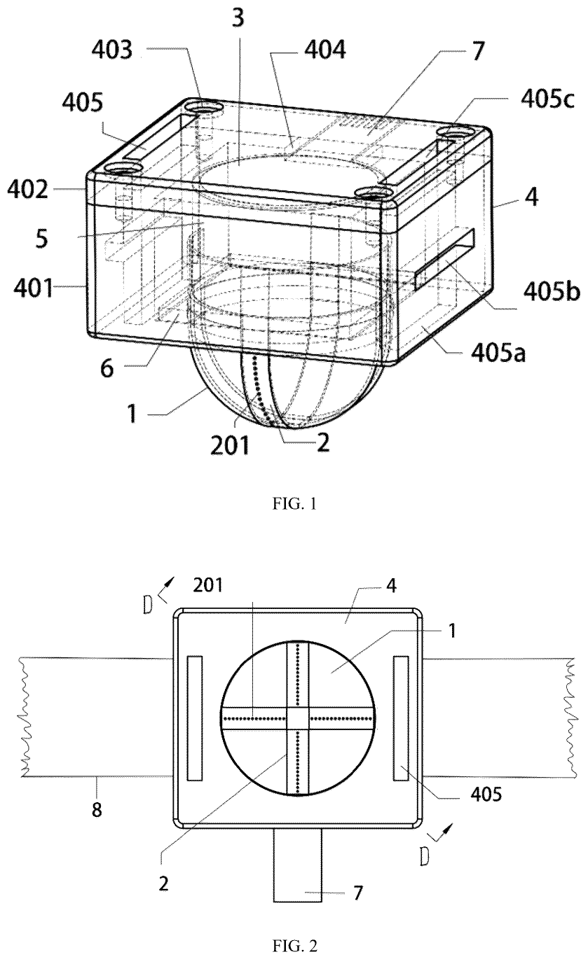

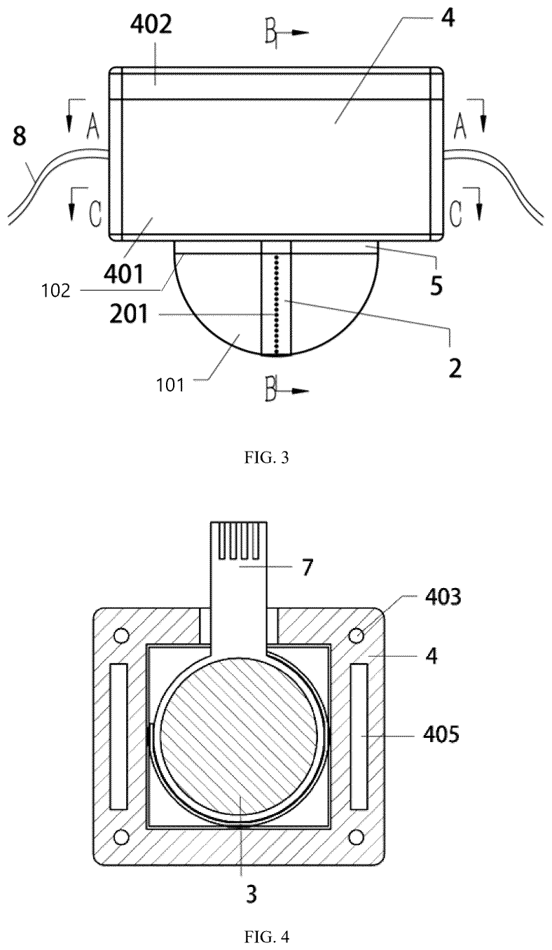

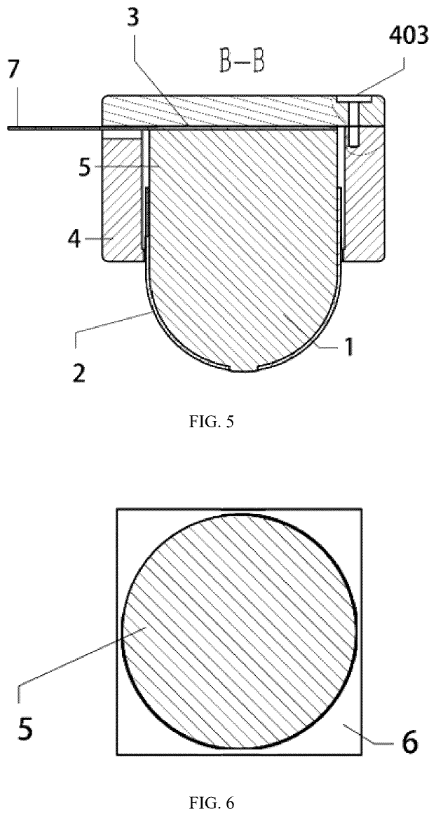

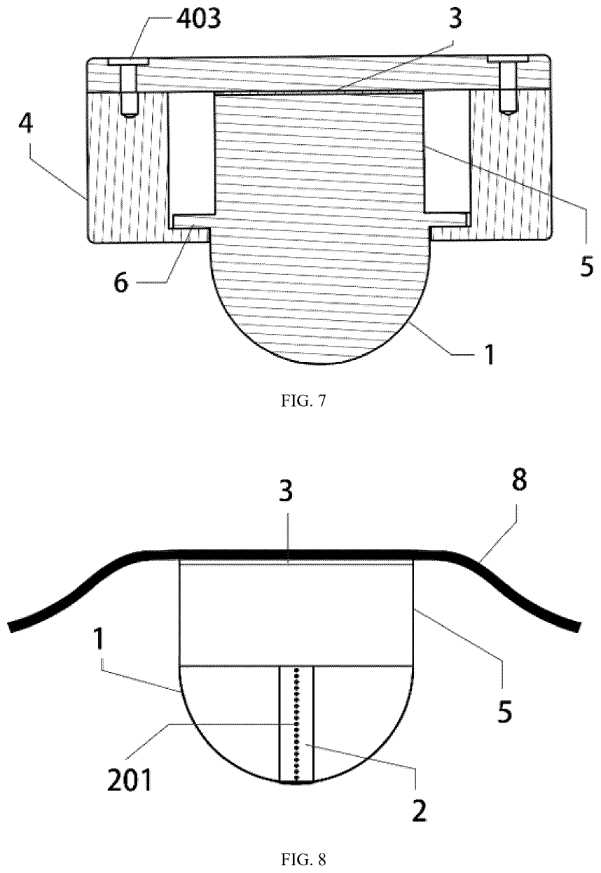

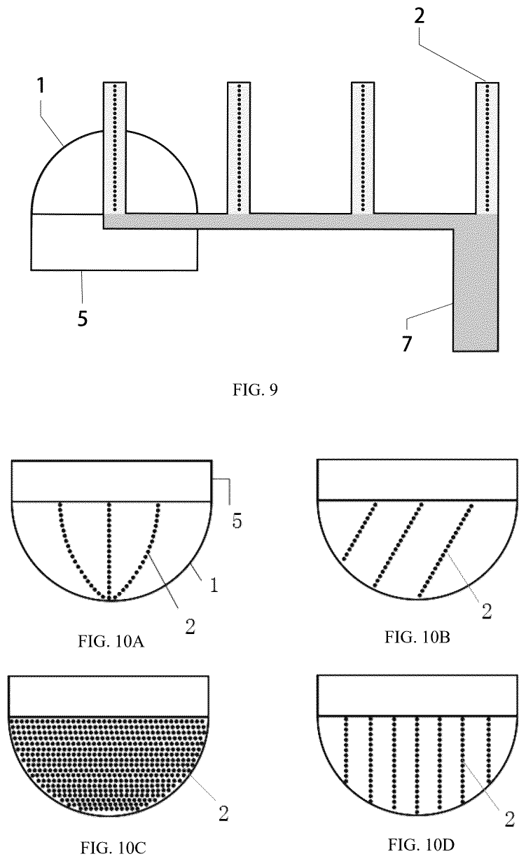

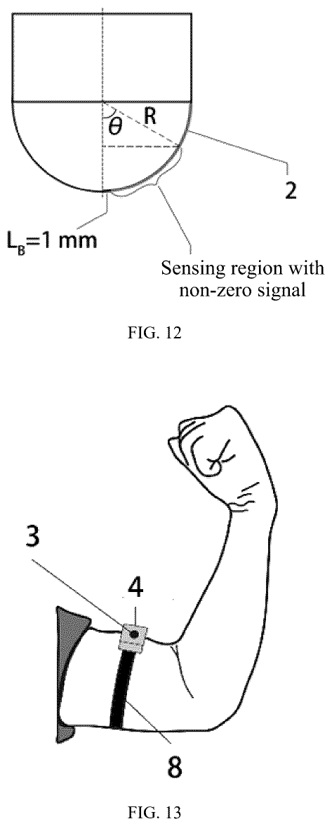

As shown in FIGS. 1 to 14, an embodiment of the present disclosure provides a wearable device for monitoring soft-tissue Young's modulus (referred to as “monitoring device”), which is mainly configured to acquire relevant data for calculating a Young's modulus of a soft tissue. The monitoring device includes indenter 1, contact radius sensor 2, contact force sensor 3, housing 4, extension column 5, wire 7, and strap 8. The housing 4 and the strap 8 collectively form a wearable mechanism for bearing the indenter 1, the contact radius sensor 2, the contact force sensor 3, and the extension column 5, etc. During use, the wearable mechanism fixes the monitoring device to a measured soft tissue region, and an indenter contact surface of a pressure application mechanism contacts the measured soft tissue, thereby enabling continuous and dynamic monitoring of the Young's modulus of the measured soft tissue.

As shown in FIG. 1, the housing 4 specifically includes a rectangular box structure formed by base 401 and top cover 402. Corresponding positions of the base 401 and the top cover 402 are each provided with screw hole 403, such that the base and the top cover are fixedly connected by screws disposed inside the screw holes 403. In other embodiments, the base and the top cover may also be fixedly connected by other detachable means such as hinging or snapping. A bottom surface of the base 401 is provided with a circular through hole with a diameter matching that of the indenter 1. A sidewall of the top cover 402 is provided with wire outlet 404 for the wire 7 to pass through. The base 401 and the top cover 402 are further provided with a plurality of strap grooves 405 for the strap 8 to pass through.

When the base 401 and the top cover 402 of the housing 4 are separated, the indenter 1 attached to the contact radius sensor 2 and the extension column 5 attached to the contact force sensor 3 are assembled into the base 401 of the housing 4. The indenter 1 and the extension column 5 may be integrally formed, or may be fixedly connected by means such as adhesion. As shown in FIG. 6, transversely protruding flange 6 is provided at a joint between the indenter 1 and the extension column 5. The shape of the flange in a top view matches the base 401 of the housing 4. The flange includes approximate isosceles triangles with a vertex angle of 90°, and has a certain thickness along a major axis of the extension column, such as 1 mm. This thickness provides necessary mechanical strength, preventing the indenter 1 from sliding infinitely outward from the base 401 of the housing 4 along the major axis of the extension column 5. In other embodiments, other means may be used to limit infinite sliding of the indenter 1 out of the base 401 of the housing. For example, a limiting mechanism disposed at a corresponding position on a sidewall of the indenter 1 or the extension column 5, or an internal structure design of the housing 4 achieves limitation after assembly. Alternatively, the extension column 5 may be directly designed as a square column with a side length of 2R, avoiding the need for an additional limiting mechanism. After assembly, the base 401 and the top cover 402 of the housing 4 are combined, and the screws are tightened into the screw holes 403 for fixation.

The strap 8 is mainly configured to fix the housing 4. Specifically, the strap 8 is fixed to the housing 4 through the strap grooves 405, and is configured to subsequently fix the monitoring device to a human tissue. Specifically, the strap 8 enters from strap groove 405b and exits through strap groove 405c. A length of the strap is adjusted until the indenter 1 is completely pressed into the measured soft tissue. Two free ends of the strap 8 are crossed and fixed above the top cover 402. In other embodiments, the fixing manner of the strap 8 may be freely adjusted, for example, the strap may enter from the strap groove 405a and exit through the strap groove 405b. The indenter 1 must be completely pressed into the measured soft tissue and must not loosen without manual intervention.

The indenter 1 is a hemispherical structure, including hemispherical contact surface 101 and top surface 102 connected to the contact surface 101. The top surface 102 is a circular flat surface. Distance D between a center point of the contact surface 101 and the top surface 102 is exactly equal to radius R of the indenter. The extension column 5 is typically a cylinder, and a diameter of the cylinder is equal to a diameter (2R) of the top surface 102. The extension column 5 is connected downward to the indenter 1 (the top surface 102) and upward to the contact force sensor 3. It is understandable that for a soft tissue with a muscle, an indentation depth is usually greater than the radius of the indenter. Through the extension column 5, the design adapts to monitoring different soft tissues, expanding the application range. The indenter 1 and the extension column 5 are integrally formed, and the formed whole may be referred to as a pressure application mechanism. In other embodiments, the indenter 1 and the extension column 5 may also be fixedly connected by means such as adhesion. Alternatively, the pressure application mechanism may only be an indenter with a hemispherical contact surface. Alternatively, the pressure application mechanism may be another structure with a hemispherical contact surface.

It should be noted that in this embodiment, a distance from a top to a bottom of the indenter is exactly equal to the radius of the indenter. Alternatively, more precisely, a chord height of the hemispherical contact surface is equal to a radius of the hemisphere. In other embodiments, the height from the top to the bottom of the indenter may be less than or greater than the radius of the indenter, forming an approximately hemispherical surface structure. However, in the present disclosure, such structure is still classified as a hemispherical surface structure. In other words, the hemisphere defined in the present disclosure may be a standard hemisphere or a non-standard approximate hemisphere. Correspondingly, the hemispherical surface structure may be a standard hemispherical surface structure or an approximately hemispherical surface structure.

The contact radius sensor 2 mainly includes one or more flexible pressure sensing arrays that can be attached to a surface of the contact surface 101 via adhesives such as double-sided tape, instant adhesive, or hot-melt adhesive. Each flexible pressure sensing array includes a plurality of pressure sensing units 201. It is understandable that a higher spatial resolution of the sensing units 201 leads to higher calculation accuracy. A smaller width of the contact radius sensor 2 provides better attachment to the contact surface 101 and is less likely to cause signal noise generated during attachment. Each sensing arrays converges through the wire and led out from the wire outlet 404 of the housing 4 for electrical connection with a data acquisition device.

The contact force sensor 3 includes a lower surface attached to a top of the extension column 5 and an upper surface contacting the housing 4 through adhesives such as double-sided tape, instant adhesive, or hot-melt adhesive. The surface shape and size of the contact force sensor 3 are the same as those of the extension column 5. The contact force sensor is also circular with a radius of R, exactly fully covering the surface of the extension column 5. In other embodiments, the surface shape and size of the contact force sensor 3 do not need to be completely the same as those of the extension column 5. For example, the contact force sensor may be circular with a radius less than R, or may be rectangular and inscribed within the surface of the extension column 5. The contact force sensor 3 is connected to the data acquisition device through the wire 7, thereby enabling signal output. The wire 7 may be led out from the wire outlet 404 of the housing 4, or may first converge with the wire connected to the contact radius sensor 2 and then be led out through the wire outlet 404.

The present disclosure imposes no special limitation on the sensing principles of the contact force sensor 3 and the contact radius sensor 2. They may be based on principles such as piezoresistive, capacitive, iontronic, or fiberoptic, with no restriction on materials of the electrode layer and functional layer, as long as pressure sensing is achievable. In this embodiment, the contact radius sensor 2 is based on the iontronic sensing principle, and its flexible pressure sensing array mainly includes an electrode layer, a functional layer, and an encapsulation layer. The electrode layer is fabricated by processing a copper foil on a flexible polyimide (PI) substrate. The functional layer is a flexible and conductive ionic membrane, mainly including thermoplastic polyurethane (TPU), ionic liquid, and indium tin oxide (ITO). The encapsulation layer is made of a flexible polyurethane (PU) material. In a preferred embodiment, the contact radius sensor includes more than two flexible pressure sensing arrays. In each flexible pressure sensing array, an extension line from an end intersects at a center point of the contact surface, while the other end converges with other flexible pressure sensing arrays through the wire for output. The design forms an overall distribution with equal arc angles about the center point of the hemispherical contact surface. Length L of each flexible pressure sensing array covers the hemispherical surface of the indenter as much as possible, and width W is generally no greater than 3 mm. Each flexible pressure sensing array includes a plurality of sensing units arranged at equal intervals (equally spaced in a flat pattern, i.e., equally spaced in the arc angle direction). Spacing D_{R} between adjacent sensing units is less than 1 mm. As shown in FIG. 2, a sensing blind zone with an arc length (L_{B}) of 1 mm is formed between a bottom end of the flexible pressure sensing array and the center point of the contact surface 101. It is understandable that pressure within the sensing blind zone is generally non-zero during actual use, so sensing units may not be arranged here. During use, at a position where the indenter 1 (the contact surface 101) contacts the measured soft tissue and generates pressure, the functional layer material of the contact radius sensor 2 is deformed, causing a capacitance value or impedance value of the sensing units to change, thereby outputting a first electrical signal. The first electrical signal is received by the data acquisition device and transmitted to a host computer. The host computer calculates a contact radius by counting a number of the sensing units with the first signal exceeding a set threshold.

It is worth noting that the contact radius sensor is based on the iontronic principle because, compared to other types of sensors, iontronic sensors have higher sensitivity and lower detection limits. They can generate the first electrical signal under smaller pressures (below 1 kPa), facilitating the capture of slight pressure signals at the contact edge between the indenter 1 and the measured soft tissue. Meanwhile, benefiting from the high sensitivity of iontronic sensors, lower pressure signals can be captured by smaller-sized sensing units. Therefore, the spatial density (resolution) of the sensing array can be increased, further ensuring the high accuracy of the contact radius sensor.

Taking a flexible pressure sensing array as an example, when some sensing units of the flexible pressure sensing array are subjected to pressure, the first electrical signal (e.g., capacitance value) output by the sensing units is changed. The first electrical signal is transmitted to the host computer via the data acquisition device. The host computer counts the number N of the sensing units with the first electrical signal exceeding the set threshold and calculates the contact radius R_{C} as follows:

$${R}_{C} = R * sin θ \qquad ( 1 )$$

$$where θ = π * \frac{{L}_{B} + 0.5 * {D}_{R} + ( N - 1 ) * {D}_{R .}}{π * R} \qquad ( 2 )$$

Thus, the contact radius is calculated as follows:

$${R}_{C} = R * {sin}^{- 1} \frac{{L}_{B} + ( N - 0.5 ) * {D}_{R}}{R} \qquad ( 3 )$$

In the equation, θ denotes the arc angle of a contact region; R denotes the radius of the indenter 1, i.e., the radius of the hemispherical contact surface 101; L_{B} denotes an arc length of the sensing blind zone; and D_{R} denotes the spacing between adjacent sensing units.

During dynamic activities, the angle between the monitoring device and the measured soft tissue may change. Therefore, the R_{C} values obtained by the four sensing arrays can be averaged. The resulting average contact radius is used as the final total contact radius for calculating the Young's modulus, thereby reducing calculation errors caused by tilting of the monitoring device.

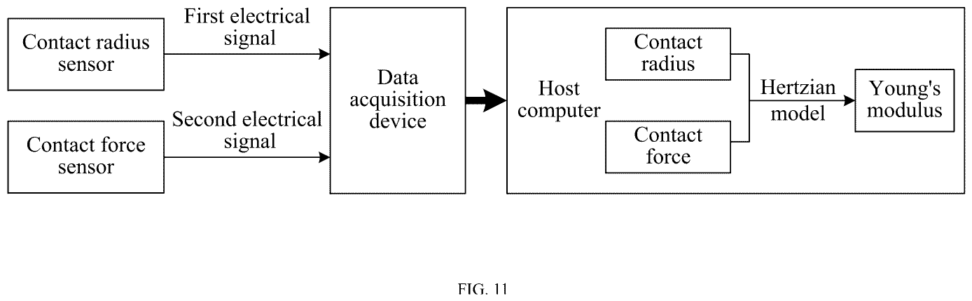

It is worth noting that the present disclosure imposes no special limitation on the arrangement of the flexible pressure sensing arrays. FIGS. 10A-10D provides several optional arrangement methods. In FIG. 10A, the distribution of the flexible pressure sensing arrays is similar to that in the aforementioned embodiment. Each flexible pressure sensing array is distributed about the center point of the hemispherical contact surface with equal arc angles, but the arc angles are smaller, for example, 45°. In FIG. 10B, each flexible pressure sensing array has an equal angle less than 90° to the horizontal plane. Both ends of the flexible pressure sensing array are close to the top surface of the indenter, with one end converging with other flexible pressure sensing arrays through the wire for output. In FIG. 10C, each flexible pressure sensing array has an approximately circular ring design (with a gap on the ring). One end of the approximately circular ring converges with other flexible pressure sensing arrays through the wire for output. The projection of the center of the approximately circular ring on the horizontal plane coincides with the center point of the contact surface. The flexible pressure sensing arrays are arranged at equal intervals along the height direction of the indenter. Alternatively, adjacent flexible pressure sensing arrays have equal arc lengths along the height direction of the indenter. In FIG. 10D, each flexible pressure sensing array has an angle of 90° to the horizontal plane, and both ends of the flexible pressure sensing array are close to the top surface of the indenter. It can be seen that the arrangement of the flexible pressure sensing arrays themselves or the sensing units within the flexible pressure sensing arrays need to detect the pressure distribution along the height direction of the indenter. Understandably, these embodiments can be divided into two categories. In one category, the sensing units within the flexible pressure sensing arrays detect the pressure distribution along the height direction of the indenter, as shown in FIG. 10A, FIG. 10B, and FIG. 10D. In the other category, the arrangement of the sensing units within the flexible pressure sensing arrays does not detect the pressure distribution along the height direction. Instead, the arrangement of a plurality of flexible pressure sensing arrays detects the pressure distribution along the height direction, as shown in FIG. 10C. The first electrical signal output by the flexible pressure sensing array actually refers to the electrical signals output by each sensing unit, meaning the first electrical signal output by the contact radius sensor actually includes a plurality of signals arranged in sequence. For these distribution designs, the host computer derives the positions of each flexible pressure sensing array or sensing unit based on a known arrangement method or sequence, determines whether the first electrical signal is greater than the set threshold, and then calculates the contact radius.

In this embodiment, the contact force sensor 3 is based on the piezoresistive principle and mainly includes an electrode layer, a functional layer, and an encapsulation layer. The electrode layer is fabricated by processing a copper foil on a PI substrate. The functional layer includes a conductive PE film. The encapsulation layer includes a PU film. The electrode layer is electrically connected to the data acquisition device through the wire 7. When the contact surface 101 of the indenter 1 contacts the measured soft tissue, a contact pressure is generated. The contact pressure is transmitted upward via the extension column 5 to the contact force sensor 3, causing deformation of the functional layer material of the contact force sensor 3 and a change in piezoresistance. An output second electrical signal is received by the data acquisition device and transmitted to the host computer. The host computer converts the second electrical signal into a corresponding contact force value. When the Young's modulus of the measured soft tissue changes dynamically, such as during muscle contraction and relaxation, the contact pressure between the indenter 1 and the measured soft tissue changes continuously and can be converted into continuous real-time contact force F. It is worth noting that the contact force sensor 3 is based on the piezoresistive principle because existing piezoresistive materials are mature in application, stable in properties, and have high accuracy, linearity, and repeatability, enabling precise capture of the contact force between the indenter 1 and the measured soft tissue.

In a specific application example, the radius R of the indenter 1 and the extension column 5 is 8 mm, and the height of the extension column 5 is 16 mm. The contact radius sensor 2 includes four pressure sensing arrays, each including 28 sensing units. In the flat pattern, the pressure sensing array has a spatial width (W) of 2 mm, a length (L) of 11.6 mm, and a thickness of 0.5 mm. The distance D_{R} (i.e., spatial resolution) between adjacent sensing units is 0.4 mm, and the arc length L_{B} of the sensing blind zone is 1 mm. The contact force sensor 3 has a radius of 8 mm and a thickness of 0.25 mm. The housing 4 has a rectangular box structure, with a height, length, width, and wall thickness being 10 mm, 26 mm, 26 mm, and 3 mm, respectively. The strap grooves 405 of the housing 4 have the same shape and size, with a length of 16 mm and a width of 2 mm.

It is worth noting that in practical use, the above dimensions can be flexibly adjusted according to requirements. For example, the radius of the indenter 1 and the extension column 5 may take any value greater than 0. The spatial size of the pressure sensing array of the contact radius sensor 2 can be flexibly adapted to the radius of the indenter 1, and its spatial resolution can be flexibly adjusted according to detection accuracy requirements. The height of the extension column 5 can be adjusted according to the tissue thickness and detection requirements. When the height of the extension column 5 is a negative value, it indicates that the height of the indenter is less than its radius. The radius of the contact force sensor 3 may take any value greater than 0, generally not exceeding the radius of the extension column 5, and its thickness may take any value greater than 0. The size of the housing 4 can be flexibly adjusted according to the size of the indenter 1 and the flange 6. The length and width of the strap grooves 405 can be flexibly adjusted according to the size of the housing 4.

In this embodiment, the indenter 1, the extension column 5, and the housing 4 may be made of a resin material by three-dimensional (3D) printing, and the strap 8 is made of a nylon material. However, it is understandable that the present disclosure imposes no limitation on the material types of the indenter 1, the extension column 5, and the housing 4. Understandably, the material hardness needs to be significantly higher than that of the measured soft tissue, with common choices including ceramics, glass, carbon fiber, polymer, and other high-modulus composite materials. The indenter 1 and the housing 4 may be made of different or the same material. The strap 8 may also be made of materials such as nylon, polydimethylsiloxane (PDMS), silicone, PU, rubber, spandex, polyester fiber, leather, and its artificial products. It is generally in a fabric form to ensure a certain strength and necessary flexibility.

In the present disclosure, the shape of the housing 4 may take various forms, such as a cylinder or a box structure with rounded corners. The connection means between the housing 4 and the strap 8 can also be flexibly adjusted, not necessarily the connection means via the strap grooves 405. For example, it may be connected by means such as buckles or adhesion. In other embodiments, the monitoring device may also be configured without the housing 4. For example, the strap 8 may directly cover the pressure application mechanism (the contact force sensor 3), but this means may require calibration for specific application scenarios. For example, when the monitoring device is fixed to the biceps brachii, a pressure is applied above the strap 8 using a calibration device (e.g., an inductance-capacitance-resistance meter (LCR)). The force value output by the calibration device and the second electrical signal output by the contact force sensor 3 are recorded to establish a fitting relationship between them. A calibration curve describing this fitting relationship is obtained for subsequent signal conversion.

Furthermore, the embodiment of the present disclosure further provides a wearable system for monitoring soft-tissue Young's modulus (referred to as “monitoring system”), mainly including the wearable device for monitoring soft-tissue Young's modulus, a data acquisition device, and a host computer. The monitoring device may adopt the structure described in the foregoing embodiments. The data acquisition device mainly includes an electrical signal acquisition circuit and a communication module. It is electrically connected to the wearable device for monitoring soft-tissue Young's modulus through the wire and is mainly configured to acquire and transmit electrical signals. The data acquisition device can be worn on a human body, and its spatial distance from the monitoring device is generally within 10-30 cm. In a specific application example, the measured soft tissue is the biceps brachii. The data acquisition device may be fixed to a position above a shoulder or a back via a tape or strap, or fixed to a waist via a belt. The data acquisition device can communicate with the host computer via Bluetooth or wireless transmission, etc. Correspondingly, the communication module may be a Bluetooth module, a WIFI module, etc.

In another embodiment, the present disclosure may also directly integrate the data acquisition module inside the housing 4 as part of the monitoring device, based on the monitoring device described in the foregoing embodiments. The housing 4 does not need the wire outlet 404. Instead, inside the housing 4, the contact radius sensor 2 and the contact force sensor 3 are directly electrically connected to the data acquisition module, respectively.

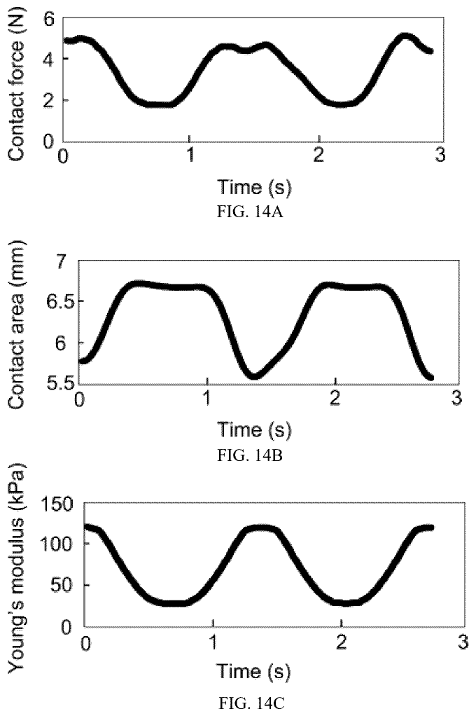

As shown in FIG. 13, during measurement, the strap 8 encircles a measurement site. The housing 4 is pressed to attach the base 401 of the housing maintain to the skin surface, causing the indenter 1 to be completely pressed into the measured soft tissue. The strap 8 is fixed to the arm. Therefore, wearable continuous and dynamic monitoring of the soft-tissue Young's modulus is achieved. That is, during any dynamic activity of the human body, the first electrical signal and the second electrical signal are continuously acquired and transmitted to the host computer via the data acquisition device. The host computer calculates curves of the contact force and the contact radius between the indenter 1 and the measured soft tissue changing over time (as shown in FIGS. 14A-14C). Based on a Hertzian contact model, an equivalent Young's modulus value of the measured soft tissue is calculated, yielding a curve of the equivalent Young's modulus value changing over time:

$$E = \frac{3 FR ( 1 - {v}^{2} )}{4 {{R}_{CA}}^{3}} \qquad ( 4 )$$

In the equation, E denotes the equivalent Young's modulus of the measured soft tissue; F denotes the contact force, converted from the second electrical signal acquired by the contact force sensor; R denotes the radius of the indenter, with a known value; v denotes a Poisson's ratio of the measured soft tissue, with a known value of 0.45; RCA denotes a total contact radius between the measured soft tissue and the indenter, calculated from the first electrical signal acquired by the contact radius sensor.

It is understandable that the host computer in the present disclosure may be a mobile terminal device such as a smartphone, a smartwatch, a smart bracelet, a tablet computer, or a laptop computer, or a desktop computer, or a dedicated terminal device in the medical field, as long as it can implement data processing and calculation functions. The host computer at least includes a calculation module, a storage module, and a communication module. The communication module is communicatively connected to the data acquisition device via wireless communication to receive the first electrical signal acquired by the contact radius sensor 2 and the second electrical signal acquired by the contact force sensor 3 from the data acquisition device. The calculation module is a general and/or specialized processing module with processing and calculation capabilities. It can call programs pre-stored in the storage module and execute corresponding instructions to implement the calculation process of the Young's modulus.

Finally, it should be noted that it is obvious to those skilled in the art that the present disclosure is not limited to the details of the above exemplary embodiments, and that the present disclosure can be implemented in other specific forms without departing from the spirit or basic features of the present disclosure. Accordingly, the embodiments should be regarded in all points of view as exemplary and not restrictive, the scope of the present disclosure being defined by the appended claims rather than the foregoing description, and it is therefore intended that all changes falling within the meaning and scope of equivalent elements of the claims should be included in the present disclosure. The reference numerals in the claims should not be considered as limiting the involved claims.

1. A wearable device for monitoring soft-tissue Young's modulus, comprising: a pressure application mechanism, a contact radius sensor, a contact force sensor, and a wearable mechanism, wherein
the pressure application mechanism comprises an indenter, and the indenter is provided with a hemispherical contact surface;
the wearable mechanism is configured to carry the pressure application mechanism and to bring the hemispherical contact surface into contact with a measured soft tissue when the wearable mechanism is worn;
the contact radius sensor comprises a flexible pressure sensing array disposed on the hemispherical contact surface and configured to detect a pressure distribution along a height direction of the indenter;
the contact radius sensor is configured to output a first electrical signal in response to a contact pressure when the hemispherical contact surface contacts the measured soft tissue; and
the contact force sensor is disposed on the pressure application mechanism and configured to output a second electrical signal in response to the contact pressure provided by the pressure application mechanism when the hemispherical contact surface contacts the measured soft tissue.

2. The wearable device according to claim 1, wherein there is at least one flexible pressure sensing array; and each of the at least one flexible pressure sensing array comprises a plurality of sensing units configured to detect the pressure distribution along the height direction of the indenter.

3. The wearable device according to claim 2, wherein there are at least two flexible pressure sensing arrays; and an extension line of an end of each of the at least two flexible pressure sensing arrays intersects at a center point of the hemispherical contact surface.

4. The wearable device according to claim 1, wherein a plurality of flexible pressure sensing arrays are arranged at equal intervals or equal arc lengths along the height direction of the indenter.

5. The wearable device according to claim 4, wherein each of the plurality of flexible pressure sensing arrays comprises a plurality of sensing units distributed circumferentially along the hemispherical contact surface.

6. The wearable device according to claim 1, wherein the pressure application mechanism further comprises an extension column fixedly connected to or integrally formed with the indenter; and the contact force sensor is disposed at an end of the extension column, wherein the end of the extension column is away from the indenter.

7. The wearable device according to claim 1, wherein the wearable mechanism comprises a housing and a strap connected to the housing; the housing is provided with a through hole; and the pressure application mechanism is disposed inside the housing, and the hemispherical contact surface of the pressure application mechanism extends out of the housing via the through hole.

8. The wearable device according to claim 7, wherein the contact force sensor comprises a first surface attached to the pressure application mechanism and a second surface opposite to the first surface and contacting an inner surface of the housing.

9. The wearable device according to claim 7, further comprising a limiting mechanism, wherein an extension column is a cylinder with a diameter equal to a diameter of the hemispherical contact surface of the indenter; and the limiting mechanism is configured to prevent the pressure application mechanism from disengaging from the housing via the through hole.

10. The wearable device according to claim 9, wherein the limiting mechanism is a flange transversely protruding from a joint between the indenter and the extension column or a limiting member disposed on the pressure application mechanism.

11. The wearable device according to claim 1, wherein the wearable mechanism is a strap; and when the indenter contacts the measured soft tissue, the strap contacts the contact force sensor.

12. The wearable device according to claim 1, wherein the contact radius sensor is an iontronic sensor.

13. The wearable device according to claim 1, wherein the contact force sensor is a piezoresistive sensor.

14. The wearable device according to claim 1, further comprising a data acquisition module communicatively connected to a host computer, wherein the contact radius sensor and the contact force sensor are electrically connected to the data acquisition module via wires, respectively.

15. A wearable system for monitoring soft-tissue Young's modulus, comprising: a host computer, and the monitoring device according to claim 1, and
alternatively, comprising the host computer, a data acquisition device, and the monitoring device according to claim 1, wherein the data acquisition device is communicatively connected to the host computer; and the contact radius sensor and the contact force sensor are electrically connected to the data acquisition device via wires, respectively; and
the host computer comprises a calculation module, a storage module, and a communication module; and the calculation module is configured to call a program pre-stored in the storage module and execute a following instruction:
acquiring the first electrical signal and the second electrical signal received by the communication module;
calculating a contact radius between the hemispherical contact surface and the measured soft tissue based on the first electrical signal;
converting the second electrical signal into a contact force between the hemispherical contact surface and the measured soft tissue; and
calculating an equivalent Young's modulus value of the measured soft tissue based on a Hertzian equation, the contact radius and the contact force.

16. A method for wearably monitoring soft-tissue Young's modulus, comprising:
acquiring a first electrical signal output by a flexible pressure sensing array in a contact radius sensor in response to a contact pressure when a contact surface of the pressure application mechanism contacts a measured soft tissue, wherein the contact surface is hemispherical; and the flexible pressure sensing array is disposed on the contact surface and configured to detect a pressure distribution along a height direction of an indenter;
calculating a contact radius between the contact surface and the measured soft tissue based on the first electrical signal;
acquiring a second electrical signal output by a contact force sensor in response to the contact pressure provided by the pressure application mechanism when the contact surface of the pressure application mechanism contacts the measured soft tissue, wherein the contact force sensor is disposed on the pressure application mechanism;
converting the second electrical signal into a contact force between the contact surface and the measured soft tissue; and
calculating an equivalent Young's modulus value of the measured soft tissue based on a Hertzian equation, the contact force and the contact radius.

17. The method for wearably monitoring soft-tissue Young's modulus according to claim 16,
wherein when the flexible pressure sensing array comprises a plurality of sensing units configured to detect the pressure distribution along the height direction of the indenter, the calculating the contact radius between the contact surface and the measured soft tissue based on the first electrical signal comprises:
counting a number of sensing units with the first electrical signal exceeding a set threshold in the flexible pressure sensing array; and
calculating the contact radius between the contact surface and the measured soft tissue based on the number and positions of the sensing units.

18. The method for wearably monitoring soft-tissue Young's modulus according to claim 17,
wherein when a plurality of flexible pressure sensing arrays are arranged, the calculating the equivalent Young's modulus value of the measured soft tissue based on the Hertzian equation, the contact force and the contact radius comprises:
averaging contact radii corresponding to the plurality of flexible pressure sensing arrays to obtain an average value, and taking the average value as a total contact radius between the contact surface and the measured soft tissue; and
calculating the equivalent Young's modulus value of the measured soft tissue based on the Hertzian equation, the contact force and the total contact radius.

19. The method for wearably monitoring soft-tissue Young's modulus according to claim 17, wherein when an extension line of an end of the flexible pressure sensing array coincides with a center point of the contact surface, a sensing blind zone is formed in a center point region of the contact surface; and when the flexible pressure sensing array comprises a column of sensing units distributed at equal arc lengths along the height direction of the indenter, the contact radius between the contact surface and the measured soft tissue is calculated as follows:
$${R}_{C} = R * {sin}^{- 1} \frac{{L}_{B} + ( N - 0.5 ) * {D}_{R}}{R}$$
wherein N denotes the number of sensing units with the first electrical signal exceeding the set threshold; R denotes a radius of the indenter; L_{B} denotes an arc length of the sensing blind zone; and D_{R} denotes a spacing between adjacent sensing units.

20. The method for wearably monitoring soft-tissue Young's modulus according to claim 16, wherein when a plurality of flexible pressure sensing arrays are arranged at equal intervals or equal arc lengths along the height direction of the indenter, and each of the plurality of flexible pressure sensing arrays comprises a plurality of sensing units arranged in an array, the calculating the contact radius between the contact surface and the measured soft tissue based on the first electrical signal comprises:
calculating an average value of the first electrical signal corresponding to each of the sensing units in each of the plurality of flexible pressure sensing arrays;
counting a number of the first electrical signal with the average value exceeding a set threshold; and
calculating the contact radius between the contact surface and the measured soft tissue based on the number and positions of the plurality of flexible pressure sensing arrays.
